(19) United States
(12) Patent Application Publication
(10) Pub. No.: US 2025/0223248 A1
(43) Pub. Date: Jul. 10, 2025

(54) METHOD FOR CONTROLLING A SYNTHESIS LOOP

(71) Applicant: CASALE SA, Lugano (CH)

(72) Inventors: Ermanno FILIPPI, Castagnola (CH); Sergio PANZA, Como (IT); Maurizio RIZZI, Villa Guardia (IT); Leonardo Angelo GALIMBERTI, Vercana (CO) (IT)

(21) Appl. No.: 18/853,500

(22) PCT Filed: Mar. 29, 2023
(86) PCT No.: PCT/EP2023/058158

§ 371 Date: Oct. 2, 2024

(30) Foreign Application Priority Data
Apr. 6, 2022 (EP) 22167015.1

(51) Int. Cl. C07C 29/152; C01C 1/04

(52) U.S. Cl. CPC C07C 29/152; C01C 1/047; C01C 1/0482

(57) ABSTRACT

A method for controlling a synthesis loop for production of a chemical product, such as ammonia or methanol, wherein a feed rate of the loop depends on at least one source of renewable energy, the pressure of the loop and the delivery rate of a makeup compressor and of a circulation compressor are continuously controlled to follow the variable rate of the feed while maintaining the loop pressure within a target deviation from a design loop pressure.

### FIELD OF THE INVENTION

The present invention relates to the production of a chemical product, like for example ammonia or methanol, wherein a suitable makeup gas is reacted in a synthesis loop under a loop pressure.

### PRIOR ART

The industrial production of various chemical products is based on reacting a makeup gas in a so-called synthesis loop. The loop includes at least a synthesis converter, which is usually a catalytic converter, wherein the makeup gas is reacted under suitable conditions of pressure and temperature. The effluent of the converter typically contains unconverted reagents which are separated and recycled to the converter, thus forming the loop. Notable examples are the systems for production of ammonia and methanol.

In most cases of practical interest, the synthesis requires a high pressure. Typically, the synthesis pressure is much higher than the pressure at which the makeup gas is produced. For example in an ammonia synthesis plant, the makeup gas may be produced in a front-end at a pressure around 25-45 bar whereas the loop pressure is well above 100 bar. Therefore the makeup gas is elevated to the loop pressure by a suitable makeup compressor. A second compressor, called circulation compressor, is used to maintain circulation in the loop, i.e. to compensate for the pressure losses, due to the partial conversion of the reactants, in the items and piping of the loop.

The synthesis loop is normally designed to operate with a constant or nearly constant rate of makeup gas feed. This is because the makeup gas is conventionally produced in a front-end running on fossil fuel (e.g. natural gas) which is constantly available and the entire plant is not designed to be flexible, relying on a nearly constant input of makeup gas. Accordingly the above mentioned compressors can be operated at a substantially constant speed of rotation and delivery rate.

In recent years, the need to reduce CO2 emissions and replace fossil fuels with renewable energy sources has emerged. For example, many processes of interest use a hydrogen-containing makeup gas and hydrogen can be produced by electrolysis of water powered with a renewable source, e.g. solar, wind, and others.

However renewable source is intrinsically intermittent and/or variable so that the rate of makeup gas feed, when dependent partially or in full from a renewable energy source, becomes variable.

Controlling a synthesis loop under a highly variable feed rate poses various challenges. For example the flow rate of a compressor may be controlled by varying the speed of rotation, however this is possible only within certain limits to ensure safe operation of the compressor. In a centrifugal compressor, reducing the speed of rotation would also decrease the delivery pressure. In some embodiments, the circulation compressor and the makeup compressor are mounted on the same shaft, so that reducing the speed of the circulation compressor to reduce the flow rate in the loop would also slow down the makeup compressor, lowering the loop pressure at inacceptable level.

If the pressure falls below a certain level, the efficiency of conversion may be severely affected or the reactor may be unable to sustain the reaction of conversion. A significant change in the loop pressure may also affect equilibrium conditions in other equipment of the loop, such as the equilibrium in a condenser. Furthermore, fluctuation of the loop pressure following the fluctuation of the renewable power may rapidly damage the items of the loop, e.g. the pressure vessel of the synthesis reactor, due to fatigue stress.

For the above reasons, the problem of how to control a synthesis loop in the presence of a highly variable feed, due to fluctuation of input power, is not easy to solve.

A control of an ammonia synthesis loop at partial load is disclosed in EP 3 819 261. A method for the control of a loop for preparation of ammonia or methanol is described in WO 2021 233 780.

### SUMMARY OF THE INVENTION

The invention faces the problem of how to control a synthesis loop for production of a chemical product, particularly for ammonia or methanol, wherein the feed of the loop depends on renewable energy and, consequently, the loop must be controlled to properly follow the fluctuation of said energy.

The problem is solved with a method of claim 1, wherein the pressure of the loop and the delivery rate of said makeup compressor and of said circulation compressor are continuously controlled to follow the variable rate of makeup gas while maintaining the loop pressure within a target deviation from a design loop pressure, wherein said target deviation is no more than 20%. Preferred embodiments are described in the dependent claims.

In the invention, the loop is dynamically controlled to react to the variation of the feed, induced by the fluctuating power of the renewable source, while maintaining the pressure loop close to the design value. The related advantages include a more stable and more reliable operation of the loop; reduced mechanical stress; optimized production based on the available power without the need of a large buffer storage. These advantages will become more evident with the following description.

### DESCRIPTION OF THE INVENTION

The makeup compressor and the circulation compressor may be reciprocating compressors or centrifugal compressors. Said two types of compressors (make up and circulator) may share the same shaft, so that they rotate at the same speed, or may have separate shafts so that the speed of rotation of each compressor can be controlled independently.

The term compressor denotes the relevant stage(s) of compression. Typically, at least the makeup compressor is multi-stage due to the considerable compression ratio. Each stage of compression may include rotary parts such as an impeller and stationary parts such as a diffuser or stationary vanes. In embodiments with a common shaft, the makeup compressor and the circulation compressor may be regarded as parts of a single machine and may have various structural parts or ancillary equipment in common.

In the invention, the loop pressure is kept within a restricted range of values independently from the loop load by controlling the delivery rate of the compressors through systems like the rotating speed, or by acting on their control systems, like suction valves or other means as described below.

The loop load is measured as the amount of fresh make-up gas fed to the synthesis loop. The loop pressure denotes the operating pressure of the ammonia converter.

### Reciprocating Circulation Compressor

The circulation compressor may be a reciprocating compressor, i.e. a positive displacement compressor, typically using one or more pistons mounted on a crankshaft.

The delivery rate of a reciprocating circulation compressor is controlled preferably by means of one or more of the following means, including combinations thereof:
a) one or more suction valve unloaders;
b) one or more suction valve modulators;
c) one or more fixed or variable clearance pockets;
d) a bypass system integrated in the compressor;
e) an independent flow throttling valve arranged to control the flow rate in the loop.

A suction valve unloader according to a) is an actuator device arranged to keep, if activated, the compressor suction valve(s) fully open even during the compression phase. The compressor may include a plurality of such suction valve unloaders so that activating them will reduce at steps the delivery rate of the compressor. A suction valve unloader acts on-off on one or more respective suction valves.

A suction valve modulator according to b) is an actuator device having the purpose to modulate the degree of opening of the suction valves during the compression phase. By controlling this device, the delivery rate can be adjusted continuously.

Fixed or variable clearance pockets according to c) are devices arranged to add clearance to the cylinder(s) of a reciprocating compressor, thus varying the quantity of gas actually delivered by each cylinder at each compression phase and, consequently, the delivery rate for a given speed of rotation.

A bypass system according to d) is a system that can recirculate a portion of the flow rate from the discharge side to the suction side.

A flow throttling valve according to e) may be located in principle at any point in the loop, due to the closed configuration of the loop itself. However, a preferred location of said flow throttling valve is at the suction of the circulation compressor and even more preferably immediately upstream the circulator compressor, wherein the term immediately upstream denotes that no equipment (such as a heat exchanger) is installed between the valve and the compressor.

The above systems a) to e) may be adopted singularly or in combination according to different embodiments. Furthermore the above systems a) to d) may be combined with controlling the speed of rotation of the compressor. In case of a reciprocating compressor, the speed of rotation refers to rotation of the crankshaft.

In some embodiments both the makeup compressor and circulation compressor are reciprocating machines. In this embodiment the two said compressors share the same shaft so that they are arranged to rotate always at the same speed. In such a case, the method may comprise controlling the speed of rotation of both compressors by controlling the speed of the common shaft.

In embodiments with reciprocating compressors on the same shaft, controlling the common speed of rotation can adjust the flow rate according to the available feed of makeup gas, whereas it does not affect substantially the loop pressure, which is determined by the compression ratio of the makeup compressor.

### Centrifugal Circulation Compressor

In certain embodiments the circulation compressor may be a centrifugal compressor, including rotating vaned impellers.

According to preferred embodiments the delivery rate of the circulation compressor, when the compressor is centrifugal, is controlled by one or more of the following means, or combinations thereof:
a) controlling the position of inlet guide vanes of the compressor;
b) controlling the opening of a rate control valve installed at suction or anyway in the loop upstream to the circulator compressor
c) controlling the speed of rotation of the compressor;

Inlet guide vanes according to a) are adjustable stationary vanes at the inlet side of the compressor, which may be arranged to impart a suitable direction or swirl to the inlet flow. By adopting adjustable guide vanes and by controlling their position (also termed degree of opening), the inlet flow can be modified, thus modifying the operation of the compressor and the flow rate that can be discharged by the same.

A control valve according to b) is a valve placed on the suction side of the compressor, which may introduce a variable pressure drop at the inlet of the compressor and, accordingly, may reduce the flow and increase the compression ratio across the machine.

In some embodiments, the makeup gas compressor and the circulation compressor are both centrifugal and mounted on the same shaft, so that they rotate at the same speed. However in a centrifugal compressor, contrary to a reciprocating compressor, the pressure produced at the discharge side depends strongly on the speed of rotation. This means that controlling the speed of the circulation compressor affects the speed of the makeup compressor and, consequently, the pressure loop.

To obviate the above problem, the invention provides, in a preferred embodiment, that:
the speed of rotation of the makeup compressor is controlled to maintain the loop pressure within the target range, the delivery rate of said makeup compressor being controlled separately from the speed of rotation, preferably by a kick-back or bypass system integrated in the compressor;
the delivery rate of the circulation compressor is controlled with any of the means a), b), c) mentioned above, or combinations thereof.

### Preferred Embodiments

The following is a description of preferred embodiments based on the type of machine used for the circulator and/or for the makeup gas compressor, i.e. reciprocating or centrifugal, and on the compressors being independent or driven by a common shaft.

In a first embodiment, the circulator is a reciprocating machine, the speed of rotation of said circulator is reduced when the feed decreases and is increased when the feed increases. The reduction or increase of the feed corresponds to reduction or increase of production.

In a second embodiment the circulator is a reciprocating machine and the delivery rate of said circulator is controlled with one or more of the above-mentioned system including suction valve unloader(s); suction valve modulator(s); fixed or variable clearance pockets; integrated bypass system.

In a third embodiment the circulator is a centrifugal machine separated by the makeup compressor and the speed of rotation of said circulator is reduced when the feed decreases and is increased when the feed increases. The speed of the makeup compressor is controlled independently.

In a fourth embodiment the circulator is a centrifugal machine and is controlled by closing a suction valve when the feed decreases and opening said valve when the feed increases, wherein the makeup compressor may be separated or mounted on the same shaft.

In a fifth embodiment the circulator is a centrifugal machine and is controlled by closing inlet guide vanes when the feed decreases and opening said guide vanes when the feed increases, wherein the makeup compressor may be separated or mounted on the same shaft.

In a sixth embodiment the circulator is a reciprocating machine and is controlled by reducing or increasing the speed of rotation according to a reduction or increase of the feed and, at the same time, is controlled by one or more of: suction valve unloader(s); suction valve modulator(s); fixed or variable clearance pockets; integrated bypass system, throttling or opening the independent valve installed at suction or anyway in the loop upstream to the circulator compressor.

In a seventh embodiment the circulator and the makeup gas compressor are separate centrifugal machines and the circulator is controlled by reducing or increasing the speed of rotation according to a reduction or increase of the feed and, at the same time, by controlling the opening of a suction valve and/or the position of adjustable guide vanes at the inlet of the circulator.

In an eighth embodiment the circulator is a centrifugal machine and the makeup gas compressor is a reciprocating machine, and the circulator is controlled by reducing or increasing the speed of rotation according to a reduction or increase of the feed and, at the same time, by controlling the opening of a suction valve and/or the position of adjustable guide vanes at the inlet of the circulator.

In all the above embodiments, the control is performed to maintain the loop pressure within the target range and, preferably, nearly constant or as constant as possible.

### Single Compressor Machine

In some embodiments, the makeup compressor and the circulator compressor can be realized as centrifugal stages of compression within a single machine. In such embodiments the method of the invention may include a combination of controlling the opening of a suction valve and controlling the position of adjustable guide vanes.

### Further Embodiments of the Invention

In the various embodiments of the invention, including embodiments with reciprocating or centrifugal compressors, the loop pressure is maintained within a target range. This means, particularly, that the loop pressure is controlled to avoid falling below the design pressure minus the given range in percentage.

In the invention, the pressure of the loop, the delivery rate of the makeup compressor and the delivery rate of the circulation compressor are continuously controlled to follow the variable rate of makeup gas while maintaining the loop pressure within a target deviation from an operating design loop pressure, wherein said target deviation is no more than 20%. A target deviation of no more than 20% may include the case of no variation when the instant pressure is equal to the design pressure of the loop. As the loop pressure may vary with time, the condition above requires that a deviation from loop pressure, if any, is not greater than 20%. For example, assuming the design loop pressure is 100 bar, a target deviation of no more than 20% means the pressure is controlled not to fall below 80 bar.

In preferred embodiments the target range is no more than 15% or no more than 10% or no more than 5%.

In embodiments, said target deviation may have a low limit such as 0.1% or 0.5% or 1.0%. Accordingly, the target deviation may be 0.1% to 20% or 0.5% to 20% or 1.0% to 20%, or ranges with any of the above low limits and an upper limit of any 15%, 10% or 5%.

The invention uses the operating design loop pressure as a setpoint for the method. While the loop pressure is maintained within the target range, the makeup gas feed may vary substantially. In a preferred embodiment, the makeup gas feed can vary within 10% to 100% of a design rate or within 20% to 100% of a design rate, whilst the loop pressure is maintained within the target range. In other words, the method of the invention maintains the loop pressure in the target range for a feed rate which may be as low as 10% of the design feed rate.

In certain embodiments of the invention, a storage of the makeup gas may be provided. Anyway a great advantage of the invention is the ability to follow the rapid fluctuation of the feed rate—dictated by the instant power of the renewable energy source, which means that a storage system, if any, can be small and less expensive. Particularly, a remarkable advantage of the invention is to follow the variable feed rate with substantially no or small variation of the temperature and pressure in the loop, particularly in the synthesis reactor. Hence the reactor does not suffer from pressure waves or heating cycles that may rapidly lead to failure.

The makeup gas in preferred embodiments is a hydrogen-containing gas wherein hydrogen is produced from electrolysis of water, using renewable energy. An interesting application is solar-powered electrolysis of water for the production of hydrogen-containing gas. Among the most interesting application of the invention are the control of an ammonia synthesis loop or a methanol synthesis loop.

An ammonia synthesis loop typically includes: a circulator, a catalytic converter, a condenser, a separator. The converter produces a hot ammonia-containing gaseous product which, after condensation, is separated into a liquid ammonia product and a gaseous phase recycled to the suction of the circulator. The ammonia makeup gas includes hydrogen and nitrogen in a suitable proportion; nitrogen may be obtained e.g. from an air separation unit or PSA.

A methanol synthesis loop is structurally similar to the above-described ammonia synthesis loop. In a methanol synthesis process, the makeup gas is typically a mixture of carbon monoxide and hydrogen.

A particularly preferred embodiment includes generation of hydrogen from electrolysis of water in a suitable electrolyser powered by renewable energy, e.g. solar-powered. It has to be noted that the output of the energy source may vary almost instantaneously, e.g. due to weather conditions. The electrolyser is also able to react in a short time, which is typically a matter of minutes. The loop, according to the invention, is able to react to the variable feed by maintaining a constant or nearly constant pressure, thus avoiding stress of the equipment and maintaining an acceptable efficiency of conversion.

### DESCRIPTION OF THE FIGURE

FIG. 1 is a simplified diagram of a loop for the production of a chemical product, for example ammonia or methanol, which can be controlled with the method of the invention.

FIG. 1 illustrates the following main items: makeup gas compressor MUC, circulation compressor CC, reactor R, heat exchanger HE, condenser COND and separator SEP.

A makeup gas 1 is fed to the inlet of the compressor MUC by a front-end FE. The front-end FE is operated with electric power P produced by a renewable source, such as a solar photovoltaic system PV.

The compressed makeup gas is sent to a cold side of the heat exchanger HE, where it is heated with heat removed from the hot effluent of the reactor R, and the heated gas is sent to the reactor R. For example the reactor R is a shell-and-tube equipment where the reaction is carried out in tubes filled with a suitable catalyst and heat of reaction is removed by water or steam in the shell side, or a plate cooled converter where the reaction is carried out in a catalyst bed incorporating cooling plates designed to remove partly or totally the reaction heat.

In other embodiments the reactor R may be a single-bed or multi-bed adiabatic reactor with or without internal heat exchangers. The reactor R may include one or more heat exchangers in the form of tubes or plates.

The effluent of the reactor R, after a passage in the hot side of the exchanger HE, is condensed and liquid fraction LP is separated from a vapour phase V. The liquid fraction LP may represent the liquid product of the loop, e.g. liquid ammonia or liquid methanol. Said vapour phase V is partly recirculated via the circulator CC, a remaining portion PRG is vented to avoid accumulation of inert gas in the loop.

The line RG represents unreacted gas reintroduced in the loop. The discharge of the circulator CC is injected in the delivery line of the main compressor MUC, as shown.

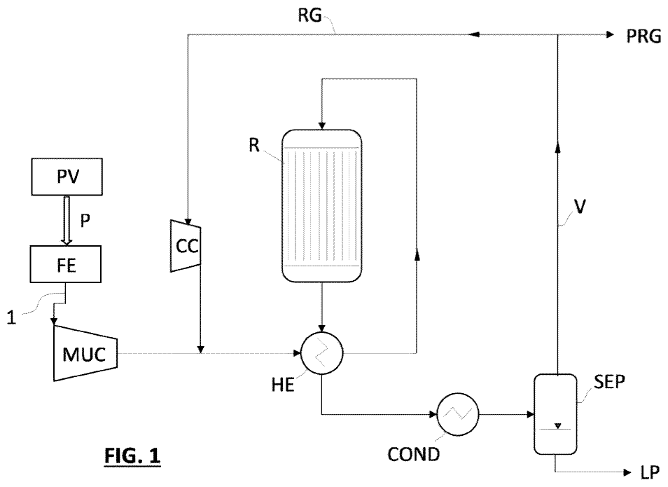

The loop of FIG. 1 reflects a typical loop for the synthesis of ammonia or methanol.

1-15. (canceled)

16. A method for controlling a synthesis loop for production of a chemical product,
wherein the synthesis loop includes at least a synthesis converter and operates at a loop pressure,
wherein production of the chemical product includes:
synthesizing said chemical product by reacting a makeup gas containing reagents in said synthesis converter,
separating a portion of unconverted reagents from an effluent of the synthesis converter, and
recycling said portion of unconverted reagents to the synthesis converter, thus forming said synthesis loop;
the method for controlling comprising:
i) elevating a pressure of the makeup gas to said loop pressure by a makeup gas compressor,
ii) maintaining circulation within the synthesis loop by a circulation compressor;
wherein a flow rate of a feed of the makeup gas depends on at least one source of renewable energy, said feed of makeup gas being time-variable as a consequence according to a time-variable power provided by said source of renewable energy; and
iii) continuously controlling:
said loop pressure,
a delivery rate of said makeup gas compressor, and
a delivery rate of said circulation compressor,
to follow the time-variable feed of makeup gas while maintaining the loop pressure within a target deviation from an operating design loop pressure, wherein said target deviation is no more than 20%.

17. The method according to claim 16, wherein step ii) comprises moving one or more pistons mounted on a crankshaft of the circulation compressor as a reciprocating compressor.

18. The method according to claim 17, wherein step iii) comprises controlling the delivery rate of said circulation compressor by one or more of the following means:
a) one or more suction valve unloaders;
b) one or more suction valve modulators;
c) one or more fixed or variable clearance pockets;
d) a bypass system integrated in the compressor;
e) controlling a speed of rotation of the crankshaft;
f) an independent flow throttling valve arranged to control the flow rate in the loop, installed at suction upstream the circulator compressor; or
g) any combination of the means a) to f).

19. The method according to claim 17, wherein the makeup gas compressor is a reciprocating compressor arranged on a same shaft of the circulation compressor so that said makeup gas compressor and said circulation compressor always rotate at a same speed,
and wherein the method further comprises controlling a speed of rotation of said compressors.

20. The method according to claim 19, wherein, specifically, controlling the speed of rotation of the compressors is combined with any of the means a) to d) and f).

21. The method according to claim 16, wherein the circulation compressor is a centrifugal compressor.

22. The method according to claim 21, further comprising controlling the delivery rate of the circulation compressor by one or more of the following means:
a) controlling a position of adjustable inlet guide vanes of the circulation compressor;
b) controlling an opening of a rate control valve placed at a suction side of the circulation compressor;
c) controlling a speed of rotation of the circulation compressor; or
d) any combination of the means a) to c).

23. The method according to claim 16, wherein the makeup gas compressor is a centrifugal compressor arranged on a same shaft of the circulation compressor so that said makeup gas compressor and circulation compressor have a same speed of rotation.

24. The method according to claim 16, wherein the makeup gas compressor is a centrifugal compressor, wherein a speed of rotation of the makeup gas compressor and a speed of rotation of the circulation compressor can be independently controlled, and wherein the method comprises:
controlling the speed of rotation of the makeup gas compressor to maintain the loop pressure within the target range, and
controlling the delivery rate of said makeup gas compressor separately from the speed of rotation of the of the makeup gas compressor by a kick-back system integrated in the compressor.

25. The method according to claim 16, wherein said target deviation of the loop pressure is no more than 10%.

26. The method according to claim 16, wherein the loop pressure is maintained within the target range while the feed of makeup gas varies within 10% to 100% of a design rate.

27. The method according to claim 16, wherein the delivery rate of the makeup gas compressor and the delivery rate of the circulation compressor are controlled separately.

28. The method according to claim 16, wherein the makeup gas is a hydrogen-containing gas.

29. The method according to claim 28, wherein hydrogen is at least partially produced by a process of electrolysis of water, which is powered by said renewable energy source.

30. The method according to claim 16, wherein the chemical product is ammonia or wherein the chemical product is methanol.
